(19) United States
(12) Patent Application Publication
(10) Pub. No.: US 2015/0050644 A1
(43) Pub. Date: Feb. 19, 2015

(54) Method for Detecting Pyrophosphoric Acid Using Support Having Boronic Acid Group Immobilized Thereon

(71) Applicant: Hitachi, Ltd., Chiyoda-ku, Tokyo (JP)

(72) Inventors: Hirokazu Nishida, Kokubunji (JP); Hideki Kambara, Hachioji (JP)

(73) Assignee: Hitachi, Ltd., Chiyoda-ku, Tokyo (JP)

(21) Appl. No.: 13/984,596

(22) PCT Filed: Dec. 5, 2012
(86) PCT No.: PCT/JP2012/081466

§ 371 Date: Aug. 9, 2013

(30) Foreign Application Priority Data
Dec. 5, 2011 (JP) 2011-265660

(51) Int. Cl. C12Q 1/68

(52) U.S. Cl. CPC C12Q 1/6811
USPC 435/6.11

(57) ABSTRACT

An object of the present invention is to provide a method capable of easily and directly performing DNA sequencing by directly detecting pyrophosphoric acid released by an enzymatic reaction, particularly with a DNA polymerase or a DNA ligase. The present invention provides a method and a device for detecting pyrophosphoric acid by measuring an electrical change occurring when a boronic acid group immobilized on an electrically conductive support reversibly binds to pyrophosphoric acid.

### TECHNICAL FIELD

The present invention relates to a method and a device for detecting pyrophosphoric acid. The invention also relates to a DNA detection method and a DNA sequencing method. More specifically, the method is configured to detect pyrophosphoric acid by detecting an electrical change occurring when pyrophosphoric acid and an electrically conductive support having a boronic acid group immobilized thereon come into contact with each other.

### BACKGROUND ART

For DNA sequencing, a method using gel electrophoresis and fluorescence detection has been widely used. In this method, many copies of a DNA fragment to be subjected to sequencing are first produced. Then, by using the 5′ end of the DNA as a starting point, fluorescently labeled fragments of various lengths are produced. At the same time, a fluorescent label having a different wavelength according to the base type at the 3′ end is attached to the 3′ end of each of these DNA fragments. These fragments are subjected to gel electrophoresis so that the difference in length is identified based on a difference by one base and luminescence emitted from each fragment is detected. From the luminescence wavelength color, the type of base at the terminal end of each DNA fragment under the measurement is found. Since DNA fragments sequentially pass through a fluorescence detection unit in ascending order of length, the type of base at the terminal end can be found in ascending order of length by measuring the fluorescent color. In this manner, sequencing is performed. A fluorescent DNA sequencer on the basis of this principle has been widely used, and also played an important role in human genome analysis (NPL 1).

On the other hand, as was declared in 2003, the human genome sequence analysis had been finished, and nowadays, sequence information has been used in the medical field as well as a wide range of industries. In light of this, in the US, for the purpose of using the human genome information in the medical field, “$1000 genome project”, which is a project for dramatically improving the cost and speed of analysis, was performed to promote the development of novel DNA analysis technique and DNA sequencer. As a result, high-speed DNA sequencers on the basis of various principles have been developed. Many of these are configured to analyze many DNA fragments in parallel using a device provided with a large number of minute reaction cells. In view of this, one of the adopted techniques is pyrosequencing using stepwise DNA complementary strand synthesis. In this method, a primer is hybridized to a target DNA strand, and four types of nucleic acid substrates for complementary strand synthesis (dATP, dCTP, dGTP, and dTTP) are sequentially added one by one into a reaction solution to perform a complementary strand synthesis reaction. When a complementary strand synthesis reaction occurs, a DNA complementary strand extends to produce pyrophosphoric acid (PPi) as a byproduct. Pyrophosphoric acid is converted into ATP by the action of a coexisting enzyme, and reacts under the coexistence of luciferin and luciferase to produce luminescence. By detecting this light, the incorporation of the added substrate for complementary strand synthesis into the DNA strand is found, so that the sequence information of the complementary strand, that is, the sequence information of the target DNA strand can be found (NPL 2).

In the originally reported pyrosequencing method, a DNA is immobilized in between a column, and a solution containing the substrates for complementary strand synthesis is allowed to flow through the column, whereby pyrophosphoric acid, which is a reaction product, is allowed to pass through several reaction sections. In this process, pyrophosphoric acid was converted into ATP, and luminescence was produced by a luminescence system using luciferin and luciferase, and the produced luminescence was detected (NPL 3, PTL 1 to PTL 3).

Further, in a method of Nyren et al., the substrates for complementary strand synthesis which were not used in the reaction were promptly degraded with an enzyme such as apyrase to eliminate the influence on the subsequent reaction step. This method may be achieved merely by sequentially adding reagents to a reaction vessel, and therefore is an easier method. In the luciferin-luciferase luminescence system, not only ATP, but also dATP, which is a substrate for complementary strand synthesis, acts on as a luminescent substrate. Thus, its analogous compound, dATP-αS, which does not serve as a luminescent substrate, is used (NPL 4, PTL 4).

Further, the present inventors have developed a method for highly sensitively examining a DNA sequence with less background luminescence, which comprises a process of producing ATP from pyrophosphoric acid and AMP using pyruvate, orthophosphate dikinase (PPDK) instead of ATP sulfurylase, which has been conventionally used as an enzyme involved in a reaction for producing ATP from pyrophosphoric acid (NPL 5). In addition, this method is suitable for performing sequence analysis of many DNA samples in parallel, and an attempt has been reported to perform parallel sequence analysis by use of several tens of thousands to several millions of reaction cells. The present inventors have also reported a method for performing sequencing of a template DNA, which comprises adjusting the concentration of substrates to be added (PTL 5).

On the other hand, recently, a technique for performing detection of a DNA or DNA sequencing using a biosensor has been reported. In the technique, a device called ISFET that detects ions is used, and a DNA probe is immobilized on a gate electrode or in the vicinity thereof of a field-effect transistor (FET), and a change in potential of the electrode when a target DNA is hybridized thereto is detected or H⁺ generated from pyrophosphoric acid which is produced as a result of DNA complementary strand synthesis is detected using a pH sensor. In an environment where a biosensor is placed, the potential of an electrode varies depending on the surface conditions. However, in the case where a large amount of salts are contained such as a bio-related solution, a change in potential sensed by the gate electrode is limited to the vicinity of the electrode (Debye length). If a spacing therebetween is larger than the Debye length, electrolytes block the electric field, and therefore, a change in potential cannot be detected. Due to this, there is a limitation on signal detection on the basis of DNA hybridization or subsequent complementary strand synthesis. On the other hand, as for the detection of H⁺ generated from pyrophosphoric acid, H⁺ can be detected using a pH sensor, and therefore, it has attracted attention as a novel DNA sequencing method. A pyrosequencing method or a method using a pH sensor is a detection method in which luminescence is produced from pyrophosphoric acid or H⁺ is generated from pyrophosphoric acid.

Pyrophosphoric acid is an important substance also from the biological point of view, and there have been reported a method in which pyrophosphoric acid is detected using luminescence (PTL 6), a method in which pyrophosphoric acid is detected by using white turbidity as an indication, an easy and highly sensitive method for specifically detecting and quantitatively determining pyrophosphoric acid by combining two enzymatic reactions using pyruvate, orthophosphate dikinase and pyruvate dehydrogenase (PTL 7), etc. However, all of these methods are not a method for directly detecting pyrophosphoric acid, and the detection process is complicated.

As a conventional method for detecting pyrophosphoric acid, a method used in a pyrosequencing method, etc. is generally known. To be more specific, the method is a method for indirectly and quantitatively measuring the release of pyrophosphoric acid, and utilizing a phenomenon, in which pyrophosphoric acid generated accompanying a DNA synthesis reaction with a DNA polymerase is once converted into ATP by the action of ATP sulfurylase, and thereafter luciferase produces luminescence using ATP, and measuring the intensity of the luminescence. In this method, since three enzymes are allowed to coexist in the same reaction vessel, the quantitative measurement performance is secured only when the concentrations of the respective substrates for the enzymes are within ranges that correspond to the equilibrium constants of the respective enzymes. That is, it is necessary to appropriately adjust the concentrations of the respective enzymes according to the amount of pyrophosphoric acid released in the early stage, and therefore, the range of the detectable concentration of pyrophosphoric acid is limited.

On the other hand, ions or anions of phosphoric acid including pyrophosphoric acid react with a compound containing a boronic acid group, and therefore a method for detecting and measuring ions or anions of phosphoric acid such as pyrophosphoric acid on the basis of produced fluorescence, which comprises fluorescently labeling the compound containing a boronic acid group and reacting the compound with a test substance to produce fluorescence has been reported (PTL 8 and PTL 9).

### CITATION LIST

### Patent Literature

PTL 1: U.S. Pat. No. 4,863,849
PTL 2: U.S. Pat. No. 4,971,903
PTL 3: U.S. Pat. No. 6,258,568
PTL 4: U.S. Pat. No. 6,210,891 (Japanese Patent No. 3510272)
PTL 5: JP-A-2007-68450
PTL 6: JP-A-2006-187251
PTL 7: JP-A-2010-200656
PTL 8: JP-A-2009-186350
PTL 9: JP-A-2001-133407

### Non Patent Literature

NPL 1: Gendai Kagaku, July 2004, vol. 400, pp. 66-69, 2004
NPL 2: Electrophoresis, vol. 22, pp. 3497-3504, 2001
NPL 3: Anal. Biochem. vol. 174, pp. 423-436, 1988
NPL 4: Anal. Biochem. vol. 242, pp. 84-89, 1996
NPL 5: Anal. Chem. vol. 78, pp. 4482-4489, 2006

### SUMMARY OF INVENTION

The method for detecting pyrophosphoric acid used in a pyrosequencing method, etc., has problems that since the reaction is performed by allowing three enzymes to coexist, and therefore, the process is complicated, and in consideration of equilibrium at the respective enzymatic reaction stages, the amount of luminescence which is the final measurement value is not in a linear relationship with the amount of pyrophosphoric acid released in the early stage. Further, in the method for detecting pyrophosphoric acid, the amount of luminescence is measured and conversion into an electrical signal is performed, and therefore, the detection result is greatly affected by the shape of the reaction cell, the amount of a solution, etc.

As described above, there have been reported only a method of indirectly obtaining DNA sequence information by undergoing a plurality of enzymatic reactions and a method which is not suitable for repetitive measurement. Therefore, a useful method for easily and directly performing sequencing by directly and quantitatively determining pyrophosphoric acid released by an enzymatic reaction, particularly with a DNA polymerase or a DNA ligase has been demanded.

The present inventors made intensive studies in order to solve the above problems, and as a result, they found that when a compound containing a boronic acid group is immobilized on an electrically conductive support and a chemical reaction that produces pyrophosphoric acid is performed on the electrically conductive support, a significant electrical change occurs in the electrically conductive support due to the presence of pyrophosphoric acid. Further, they found that by detecting pyrophosphoric acid using the electrically conductive support having a compound containing a boronic acid group immobilized thereon, DNA sequencing, detection of a DNA or an RNA, detection of a single nucleotide polymorphism, etc. can be achieved easily and efficiently. The present invention has been achieved on the basis of the above findings, and specifically includes the following aspects.

[1] A method for electrically detect pyrophosphoric acid, including:

a step of preparing an electrically conductive support having a compound containing a boronic acid group immobilized thereon;

a step of bringing a test substance into contact with a boronic acid group on the electrically conductive support;

a step of measuring an electrical change of the electrically conductive support; and

a step of detecting pyrophosphoric acid on the basis of the electrical change.

[2] The method according to [1], wherein the compound containing a boronic acid group is at least one compound selected from the group consisting of a phenylboronic acid derivative, a methylboronic acid derivative, and a propenylboronic acid derivative.

[3] The method according to [1] or [2], wherein the compound containing a boronic acid group is a water-soluble polymer molecule containing a boronic acid group.

[3-2] The method according to [3], wherein the water-soluble polymer molecule is poly((2-methacryloyloxyethyl phosphorylcholine)-co-(n-butyl methacrylate)-co-(p-vinylphenyl boronic acid)).

[3-3] The method according to [3], wherein the water-soluble polymer molecule is poly((2-methacryloyloxyethyl phosphorylcholine)-co-(dimethylaminoethyl methacrylate)-co-(p-vinylphenyl boronic acid)).

[4] The method according to any one of [1] to [3], wherein the compound containing a boronic acid group is a molecule containing a phenylboronic acid group and a thiol group.

[4-2] The method according to [4], wherein the molecule is 4-mercaptophenyl boronic acid.

[5] The method according to anyone of [1] to [4], wherein the electrically conductive support contains a noble metal.

[5-2] The method according to [5], wherein the noble metal is at least one metal selected from the group consisting of silver, gold, platinum, and palladium.

[5-3] The method according to [5-2], wherein the compound containing a boronic acid group is a molecule containing a phenylboronic acid group and a head group, and the head group is a functional group that can covalently bind to at least one metal selected from the group consisting of silver, gold, platinum, and palladium.

[5-4] The method according to any one of [1] to [4], wherein the electrically conductive support contains an electrode.

[5-5] The method according to any one of [1] to [4], wherein the electrically conductive support is placed in a solution.

[6] The method according to any one of [1] to [5], wherein the electrical change is measured by a voltage change measurement device connected to the electrically conductive support through an electrical conductor.

[6-2] The method according to [6], wherein the measurement device is a measurement device that can measure a minute voltage change.

[6-3] The method according to [6], wherein the measurement device is a measurement device having a transistor in a circuit for measuring a minute voltage change.

[7] The method according to [6], wherein the measurement device is a measurement device having a field-effect transistor (FET) in a circuit for measuring a minute voltage change.

[8] A method for sequencing a target DNA, including:

a step of detecting pyrophosphoric acid generated by DNA complementary strand synthesis with a DNA polymerase using a target DNA as a template according to the method according to any one of [1] to [7]; and

a step of determining the type of base incorporated in the DNA complementary strand synthesis on the basis of the result of detection of pyrophosphoric acid.

[8-2] A method for detecting or quantitatively determining a target DNA or RNA, including:

a step of detecting pyrophosphoric acid generated by DNA complementary strand synthesis with a DNA polymerase using a target DNA or RNA as a template according to the method according to any one of [1] to [7];

a step of determining the type of base incorporated in the DNA complementary strand synthesis on the basis of the result of detection of pyrophosphoric acid; and

a step of sequencing the whole or a part of the target DNA or RNA.

[9] The method according to [8], wherein the DNA polymerase and/or the target DNA are/is immobilized on a surface of the electrically conductive support.

[9-2] The method according to [8], wherein the DNA polymerase and/or the target DNA are/is immobilized on a surface of the electrode having the compound containing a boronic acid group immobilized thereon.

[9-3] The method according to [9], wherein the DNA polymerase to be immobilized on the electrically conductive support is a DNA polymerase having binding affinity for a boronic acid group.

[10] The method according to [9], wherein the DNA polymerase to be immobilized on the electrically conductive support is a DNA polymerase containing a peptide tag having binding affinity for a boronic acid group in its molecule.

[11] The method according to any one of [8] to [10], wherein the target DNA or RNA is immobilized on a surface of a solid support which is different from the electrically conductive support.

[11-2] The method according to [11], wherein the solid support having the target DNA or RNA immobilized thereon is placed in each of a plurality of reaction cells, an electrical change corresponding to each reaction cell is measured, and pyrophosphoric acid generated by DNA complementary strand synthesis in each reaction cell is detected.

[11-3] The method according to [11] or [11-2], wherein the solid support having the target DNA or RNA immobilized thereon is a metal bead.

[11-4] The method according to [11] or [11-2], wherein the solid support having the target DNA or RNA immobilized thereon is a magnetic bead.

[11-5] The method according to any one of [8] to [11], wherein the electrically conductive support includes a first region in which the electrode having the DNA polymerase immobilized thereon is placed and a second region which is disposed adjacent to the first region and in which a reference electrode having the compound containing a boronic acid group immobilized thereon is placed.

[11-6] The method according to [11-5], wherein in a space including the second region, an enzyme that degrades a nucleic acid synthesis substrate such as apyrase is held.

[11-7] The method according to any one of [8] to [11], wherein the sequencing of the target DNA is performed by monitoring the stepwise DNA complementary strand synthesis.

[12] A method for detecting a target DNA or RNA, including:

a step of detecting pyrophosphoric acid generated by DNA complementary strand synthesis with a DNA polymerase from a probe hybridized to the target DNA or RNA according to the method according to any one of [1] to [7]; and

a step of detecting the hybridization to the target DNA or RNA on the basis of the result of detection of pyrophosphoric acid.

[12-2] The method according to [12], wherein a gene expression profile analysis is performed by synthesizing a DNA complementary strand using each probe held in a plurality of reaction cells and detecting pyrophosphoric acid generated.

[13] A method for detecting a target DNA or detecting a single nucleotide polymorphism in a target DNA, including:

a step of detecting pyrophosphoric acid generated by ligating a nick between two probes hybridized to the target DNA by a DNA ligase reaction according to the method according to any one of [1] to [7]; and

a step of detecting the hybridization of the two probes to the target DNA on the basis of the result of detection of pyrophosphoric acid.

[14] A device for electrically detecting pyrophosphoric acid, characterized by including an electrically conductive support having a compound containing a boronic acid group immobilized thereon.

[15] The device according to [14], wherein on the electrically conductive support, further a DNA polymerase is immobilized.

[15-2] The device according to [14] or [15], which is used for DNA sequencing, detection of a target DNA or RNA, or detection of a single nucleotide polymorphism.

[16] A sensor for detecting pyrophosphoric acid, characterized by including:

an electrically conductive support having a compound containing a boronic acid group immobilized thereon; and

a unit for measuring an electrical change of the electrically conductive support.

According to the invention, a method and a device for easily and efficiently detecting pyrophosphoric acid are provided. The method and the device according to the invention directly detect pyrophosphoric acid, and therefore can achieve detection with high sensitivity and high S/N. Further, the method and the device according to the invention can be applied to every system in which pyrophosphoric acid is produced as a reaction product in various biochemical processes, and therefore provide a technique for diachronically observing the progress of such biochemical processes.

Further, by detecting pyrophosphoric acid generated by an enzymatic reaction with a DNA polymerase, a DNA ligase, or the like by utilizing the invention, DNA sequencing, detection of a target DNA or RNA, or detection of a single nucleotide polymorphism can be easily and efficiently performed.

Objects, configurations, and effects other than those described above will be apparent through the following description of embodiments.

The present specification includes the contents described in the specification, claims, and drawings of Japanese Patent Application No. 2011-265660, on which this application is based for priority.

### BRIEF DESCRIPTION OF DRAWINGS

FIG. 1(A) is a graph showing a change inpotential obtained by measurement with an FET when (1) 100 pmol of pyrophosphoric acid was added onto a gold base plate modified with phenylboronic acid, and thereafter (2) 200 pmol of pyrophosphoric acid was added thereonto. FIG. 1(B) is a graph showing a change in potential measured by an FET when (1) 100 pmol of pyrophosphoric acid was added onto an unmodified gold base plate, and thereafter (2) 200 pmol of pyrophosphoric acid was added thereonto.

FIG. 2 is a graph showing a change in potential obtained by immediate measurement with an FET when a DNA template was attached to a DNA polymerase into which a peptide tag that specifically recognizes phenylboronic acid was integrated, and a deoxyribonucleic acid (base), which is or is not a substrate, was added. First, when (a) dATP, which is not a substrate, was added, a change in potential did not occur. Then, when (b) dCTP, which can be a substrate, was added, a change in potential was observed. Further, (c) dGTP and (d) dTTP, each of which can be a substrate, were added sequentially, and a change in potential was observed as expected. At this point, the reaction was completed, and therefore, even when (e) dNTP was added in the end, a change in potential was not observed.

FIG. 3 is a view showing the result of an experiment of complementary strand synthesis with a DNA polymerase. The result obtained by adding dCTP, which is a substrate to enable a reaction, to give a final concentration of 0.05 mM using two types of template DNAs is presented as a sensorgram. The amount of change in potential in the sensorgram varied depending on the number of consecutive guanine bases (G) immediately downstream of the primer in the template DNA used. It is supposed that as the number of consecutive G bases is larger, the amount of incorporation of dCTP is increased, and therefore, the amount of released pyrophosphoric acid is also increased according to the increase. Also in the drawing, in the case of a template (a) which comprises single-base repeats (10 bases long), the amount of change was about 20 mA. However, in the case of a template (b) which comprises single-base repeats (3 bases long), the amount of change was about 6 mA.

FIG. 4(A) shows one example of a configuration of a device of the invention, and FIG. 4(B) shows one example of a configuration of a device of the invention and a reaction of pyrophosphoric acid.

### DESCRIPTION OF EMBODIMENTS

Hereinafter, the invention will be described in detail.

The invention provides a method for detecting the existence or the amount of pyrophosphoric acid by detecting an electrical change occurring when a boronic acid group (e.g., a phenylboronic acid) in a polymer immobilized on an electrically conductive support, or a boronic acid group alone immobilized on an electrically conductive support and pyrophosphoric acid come into contact with each other. The precise reaction mechanism has not been elucidated yet, but an electrical change occurs in an electrically conductive support probably due to the binding between a hydroxy group in a boronic acid group and a hydroxy group in pyrophosphoric acid. Incidentally, although each of deoxyribonucleic acids (dCTP, dATP, dTTP, and dGTP), which can be substrates for a DNA polymerase, has a hydroxy group, a significant electrical change is unexpectedly not observed in the presence of the deoxyribonucleic acids. Therefore, the invention is based on the finding that by using an electrically conductive support having a compound containing a boronic acid group immobilized thereon, pyrophosphoric acid can be specifically detected.

In the invention, an electrically conductive support having a compound containing a boronic acid group immobilized thereon is used. As the compound containing a boronic acid group, any compound can be used as long as it is a compound having a boronic acid group (a group obtained by substituting one hydroxy group of boric acid). For example, it may be an organic compound having one or more boronic acid groups, or may be a polymer compound obtained by polymerizing a monomer having a boronic acid group. Preferably, a polymer having two or more boronic acid groups is used. For example, in the case of using a polymer having a boronic acid group as one chemical group, as the overall structure of the polymer, various structures can be selected according to the property of the electrically conductive support, a method for immobilizing the polymer containing a boronic acid group, the property of a protein (such as an enzyme) to be additionally immobilized on the support, the property of a chemical reaction or an enzymatic reaction performed on the support using the same, and so on.

Specific examples of the compound containing a boronic acid group include a phenylboronic acid derivative having a structure represented by the following formula (I), a methylboronic acid derivative having a structure represented by the formula (II), and a propenylboronic acid derivative having a structure represented by the formula (III).

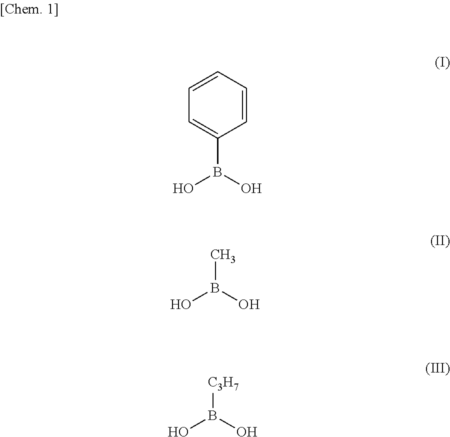

The compound containing a boronic acid group is preferably a polymer molecule, particularly a water-soluble polymer molecule. As a specific example of the polymer molecule containing a boronic acid group, for example, poly((2-methacryloyloxyethyl phosphorylcholine)-co-(n-butyl methacrylate)-co-(p-vinylphenylboronic acid)) (hereinafter abbreviated as PMBV) can be exemplified. As another specific example, poly((2-methacryloyloxyethyl phosphorylcholine)-co-(dimethylaminoethyl methacrylate)-co-(p-vinylphenylboronic acid)) (hereinafter abbreviated as PMDV), which is a polymer molecule containing a phenylboronic acid group, can be exemplified.

The compound containing a boronic acid group is preferably a molecule containing a boronic acid group and a head group reactive with the electrically conductive support. According to this, the compound containing a boronic acid group can be easily and efficiently immobilized on the electrically conductive support. The head group reactive with the electrically conductive support is preferably a head group capable of covalently binding to the electrically conductive support or a functional group introduced into the electrically conductive support, and specific examples thereof include a thiol group, an amino group, and a succinimide group.

In a preferred embodiment, the compound containing a boronic acid group is a molecule containing a phenylboronic acid group and a thiol group, for example, 4-mercaptophenyl boronic acid.

The compound having a boronic acid group may contain, in addition to a boronic acid group and a head group, one or more functional groups suitable for a binding reaction with a peptide tag useful for binding to another protein, a reaction with another compound or molecule, etc. Examples of such a group include a group which makes the local pH basic at a site where the binding reaction between a boronic acid group and a peptide tag occurs (such as a dimethylaminoethyl group or a diethylaminoethyl group) and a functional group which increases the fluidity of a compound or a molecule to increase the reaction efficiency of the compound or the molecule (such as 2-methacryloyloxyethyl phosphorylcholine).

The electrically conductive support on which a compound containing a boronic acid group is immobilized is not particularly limited as long as it is an electrically conductive support which is generally used in this technical field. Specific examples thereof include noble metals (such as gold, silver platinum, palladium, rhodium, iridium, and ruthenium); metals such as copper, aluminum, tungsten, molybdenum, chromium, titanium, and nickel; alloys such as stainless steel, hastelloy, inconel, monel, and duralumin; electrodes such as semiconductor devices (such as a transistor and an FET); silicon; glass materials such as glass, quartz glass, fused quartz, synthetic quartz, alumina, sapphire, ceramics, forsterite, and photosensitive glass; plastics such as polyester, polystyrene, polyethylene, polypropylene, nylon, acrylic resins, polycarbonate, polyethylene terephthalate (PET), polyurethane, phenol resins, melamine resin, epoxy resins, and polyvinyl chloride; agarose, dextran, cellulose, polyvinyl alcohol, and nitrocellulose. Further, a material obtained by coating a non-conductive support with an electrically conductive material can also be used as the electrically conductive support. Also the form of the electrically conductive support is not particularly limited, and a support formed of a plane (e.g., a titer plate, a porous or macroporous array, or a microchannel), a flat plate, a film, a tube, and a particle (e.g., a magnetic particle) can be exemplified. The electrically conductive support may be an electrode for easily and efficiently perform the measurement of an electrical change described below.

A method for immobilizing the compound containing a boronic acid group on the electrically conductive support is not particularly limited. For example, the compound containing a boronic acid group can be immobilized on the electrically conductive support through a covalent bond, an ionic bond, or physical adsorption. Specifically, for example, a solution (e.g., in an organic solvent such as ethanol) obtained by mixing the compound containing a boronic acid group is applied onto the electrically conductive support, followed by drying, whereby the compound containing a boronic acid group can be immobilized on the surface of the electrically conductive support. Further, in the case where the compound containing a boronic acid group has another functional group, various methods can be selected according to the property of the electrically conductive support, the property of a molecule to be additionally immobilized on the support, the property of a chemical reaction or an enzymatic reaction performed on the electrically conductive support using the same, and so on. The compound containing a boronic acid group may be bound to the electrically conductive support through a spacer sequence, for example, a hydrocarbon group having 1 to 10 carbon atoms.

The immobilization of the compound containing a boronic acid group on the electrically conductive support through a covalent bond can be performed by, for example, introducing a functional group reactive with a head group contained in the compound containing a boronic acid group into the electrically conductive support and reacting both groups with each other. In the case where the compound containing a boronic acid group has a head group capable of covalently binding to the electrically conductive support (e.g., a noble metal), by utilizing the head group, the compound containing a boronic acid group can be immobilized on the electrically conductive support of a noble metal such as gold or platinum through a covalent bond. Gold, platinum, or the like enables configuration control at an atomic level. That is, a lattice plane grown as a metal crystal can be processed into a flat plate with no irregularities at an atomic level. By forming a monolayer of the compound containing a boronic acid group thereon, a flat plate-shaped electrically conductive support for detecting pyrophosphoric acid can be obtained. That is, a device having a controlled height while controlling the orientation of molecules can be provided on this electrically conductive support made of a metal material and having a monolayer of the compound containing a boronic acid group. Further, by binding an arbitrary protein molecule to this electrically conductive support made of a metal material and having a monolayer of the compound containing a boronic acid group through a peptide tag capable of binding to a boronic acid group, a device in which the height of the molecule immobilized is controlled within an error of a few angstroms while controlling the orientation of the molecule can be provided on the metal plane. For example, on the surface of the electrically conductive support, molecules can be immobilized as a monolayer in which the molecules are held in the same orientation.

Further, for example, a covalent bond can be formed by using an amino group as the head group of the compound containing a boronic acid group, and introducing an active ester group, epoxy group, aldehyde group, carbodiimide group, isothiocyanate group, or isocyanate group into the electrically conductive support. In addition, a thiol group may be used as the head group, and an active ester group, maleimide group, or disulfide group may be introduced into the electrically conductive support. Examples of the active ester group include a p-nitrophenyl group, an N-hydroxysuccinimide group, a succinimide group, a phthalimide group, and a 5-norbornene-2,3-dicarboxyimide group. As a method for introducing a functional group into the electrically conductive support, a method in which the surface of the support is treated with a silane coupling agent having a desired functional group (such as γ-aminopropyltriethoxysilane) can be exemplified. As another method, a plasma treatment can be exemplified.

As described above, the compound containing a boronic acid group can be immobilized on the electrically conductive support. The invention also provides an electrically conductive support obtained by binding the compound containing a boronic acid group in advance to the surface of an electrically conductive solid support. This electrically conductive support can be used as a device for electrically detecting pyrophosphoric acid as described below.

In a preferred embodiment of the invention, a gold electrode is used as the electrically conductive support, and a covalent bond is formed between a thiol group and gold by allowing 4-mercaptophenyl boronic acid to act on the electrode, whereby a self-assembled monolayer (SAM) of phenylboronic acid is formed on the gold electrode so as to produce an electrode for detecting pyrophosphoric acid.

The immobilization of the compound containing a boronic acid group is not limited to the immobilization on a solid support. As another method in the invention, an electrically conductive support (immobilization base material) can be formed by crosslinking a functional group (e.g., a hydroxy group) except for a hydroxy group considered to be necessary for the compound containing a boronic acid group to detect pyrophosphoric acid using a crosslinking molecule. As a result, the compound containing a boronic acid group is immobilized on the electrically conductive support formed by crosslinking. Examples of the crosslinking molecule include polyhydric alcohols such as polyvinyl alcohol and polysaccharides such as dextran.

Examples of the electrically conductive support (immobilization base material) which can be formed include a solid, a polymer membrane, a semi-transparent polymer membrane, a film, a solid-phase gel, and a liquid-phase gel. Therefore, the invention also provides a device in which the compound containing a boronic acid group is immobilized stably through a covalent bond in a film-like substance such as a semi-transparent membrane or a gel-like substance having a different water content. Further, such an electrically conductive support can have a minute size such as a micrometer or nanometer size.

Further, by utilizing the boronic acid group immobilized in this manner, any other various molecules (such as proteins) can be immobilized on the electrically conductive support.

As described above, the compound containing a boronic acid group or other protein molecules can be bound on a noble metal support such as an electrode in an orientation-controlled manner, and therefore, it becomes possible to perform immobilization such that the active site of the immobilized compound or molecule (e.g., protein, particularly enzyme) is within a distance of about 100 Å from the surface of the noble metal support. According to this, it becomes possible to effect a chemical reaction or an enzymatic reaction (often including various charge-transfer reactions) within the Debye length of a field-effect transistor (FET) presumed to be several tens to one hundred and several tens angstroms, and therefore, a system in which a detection reaction of pyrophosphoric acid or an arbitrary enzymatic reaction is detected using an FET can be constructed. For example, a device in which the compound containing a boronic acid group is immobilized on a completely flat plate support composed of a metal single crystal while keeping the orientation in the same direction and the height at the same level is preferably used as a sensor chip such as an FET.

When a reaction that causes charge transfer occurs on a sensor chip, an FET can achieve detection with high sensitivity, but a detectable distance is supposed to be several nanometers from the surface of the support. Therefore, in the case of the electrically conductive support (e.g., a metal crystal base) having the compound containing a boronic acid group immobilized thereon while controlling the orientation as described above and keeping the height at the same level at high precision, a charge-transfer reaction can be detected effectively within a detection limit.

In the case of detecting pyrophosphoric acid, the electrically conductive support having the compound containing a boronic acid group immobilized thereon may be placed in a solution. The solution to be used is not particularly limited and can be appropriately selected according to the type of a test substance (a reaction generating pyrophosphoric acid), the type of the electrically conductive support, etc. For example, water, phosphate buffered saline (PBS), Tris buffer, or the like can be used.

Subsequently, a test substance is brought into contact with a boronic acid group on the electrically conductive support. The “test substance” as used herein refers to a substance to be detected for pyrophosphoric acid, and includes, for example, a reactant that generates pyrophosphoric acid, a reaction mixture of a chemical reaction or an enzymatic reaction, etc. Further, the “contact” refers to an operation of making the test substance and the electrically conductive support approach to each other, and this operation can be performed by, for example, adding the test substance (a liquid) onto the electrically conductive support, adding and mixing the test substance (a liquid) in a solution in which the electrically conductive support is placed, allowing a chemical reaction or an enzymatic reaction to proceed on the electrically conductive support, etc. The contact between the test substance and a boronic acid group can be performed at an appropriate temperature (e.g., 5 to 100° C.) and under an appropriate pH condition (e.g., pH 3 to 12).

Thereafter, an electrical change of the electrically conductive support is measured. The “electrical change” as used herein is known in this technical field and refers to a change in potential in the electrically conductive support and can be measured as a change in voltage or current. The electrical change can be measured by a voltage change measurement device connected to the electrically conductive support through an electrical conductor. The voltage change measurement device may be any measurement device having any circuit structure as long as it is a measurement device capable of measuring a minute voltage change, or a measurement device capable of detecting electron transfer in an electrode (a minute electric current) induced by a minute voltage change. For example, in order to measure a minute voltage change, a transistor, preferably a field-effect transistor (FET), or a measurement device having an equivalent minute electric current measurement system in a circuit can be used. In a preferred embodiment, a device structure in which an electrode is adopted as the electrically conductive support, an electrode having a boronic acid group immobilized thereon is used as a gate electrode of an FET, and an electrical change thereof can be observed over time is used. Since a change is measured, an electrical change may be measured by measuring a voltage or a minute electric current before a test substance is brought into contact, or a control voltage or a control minute electric current when a test substance is not brought into contact, and then, comparing it with a voltage or a minute electric current measured after the test substance is brought into contact.

In the case where an electrical change is measured, pyrophosphoric acid is contained in the test substance, that is, the existence of pyrophosphoric acid is detected. The degree of the electrical change varies according to the amount of existing pyrophosphoric acid, and therefore, it is also possible to quantitatively determine pyrophosphoric acid by measuring the degree of the electrical change.

In an embodiment of the invention, as the test substance, a reactant of a DNA polymerase is used. That is, pyrophosphoric acid to be detected is pyrophosphoric acid released by DNA complementary strand synthesis with a DNA polymerase using a target DNA or RNA as a template. A DNA polymerase releases pyrophosphoric acid when a deoxyribonucleic acid (a nucleotide triphosphate such as dATP, dCTP, dGTP or dTTP) complementary to the target DNA or RNA is incorporated as a substrate. Therefore, by using a target DNA whose sequence is unknown as a template, sequentially adding 4 types of deoxyribonucleic acids (substrates), and detecting pyrophosphoric acid, the presence or absence of incorporation of a substrate complementary to the sequence is determined, and in the end, it becomes possible to perform sequencing of the target DNA. Further, by monitoring the stepwise DNA complementary strand synthesis, sequencing of the whole or apart of the target DNA can be performed. Moreover, by detecting pyrophosphoric acid released by DNA complementary strand synthesis with a DNA polymerase using a target DNA or RNA as a template, the presence or absence of incorporation of a substrate complementary to the sequence is determined and sequencing of the whole or a part of the target DNA or RNA is performed, whereby the target DNA or RNA can be detected or quantitatively determined in the end.

Accordingly, the invention also relates to a method for sequencing a target DNA, and a method for detecting or quantitatively determining a target DNA or RNA.

Further, in another embodiment of the invention, a reactant of a DNA polymerase is used as the test substance, and pyrophosphoric acid to be detected is pyrophosphoric acid generated by DNA complementary strand synthesis with a DNA polymerase from a probe hybridized to a target DNA or RNA. A DNA polymerase extends a probe only when the probe is hybridized to a target DNA or RNA by performing DNA complementary strand synthesis using the target DNA or RNA as a template. It is possible to detect the target DNA or RNA by detecting pyrophosphoric acid released at this time. Further, by quantitatively detecting pyrophosphoric acid released, it is possible to measure not only the existence of the target DNA or RNA, but also the amount of the target DNA or RNA.

In an embodiment, by performing a DNA complementary strand synthesis reaction using respective probes held in a plurality of reaction cells and detecting pyrophosphoric acid corresponding to each reaction cell, target DNAs or RNAs binding to the probes are comprehensively detected, whereby a gene expression profile can be analyzed.

Accordingly, the invention also relates to a method for detecting a target DNA or RNA and a method for analyzing a gene expression profile.

The DNA polymerase is an enzyme that performs DNA complementary strand synthesis using a DNA or an RNA as a template, and is well known in this technical field. In the invention, an arbitrary DNA polymerase can be used, and an appropriate DNA polymerase can be obtained by utilizing a genetic engineering technique by those skilled in the art or as a commercially available product. Also, the target DNA or RNA is not limited, and a naturally-occurring product such as a biogenic product may be used, or a synthetic product such as a library may be used. The probe to be used varies depending on the intended purpose, but those skilled in the art can appropriately design the probe.

In the case where pyrophosphoric acid generated by a DNA polymerase reaction is detected, a DNA polymerase and/or a target DNA or RNA may be immobilized on an electrically conductive support. For example, a DNA polymerase and/or a target DNA or RNA may be immobilized on a surface of an electrode having a compound containing a boronic acid group immobilized thereon. Preferably, a DNA polymerase is immobilized on an electrically conductive support or an electrode.

In an embodiment, on an electrically conductive support to be used, a DNA polymerase is immobilized on a compound containing a boronic acid group, and the compound containing a boronic acid group is immobilized on a reference electrode (a first region). It is preferred that in a region around this electrode (a second region), an enzyme that degrades a nucleic acid synthesis substrate (such as apyrase) is held.

In another embodiment of the invention, a reactant of a DNA ligase is used as the test substance. That is, pyrophosphoric acid to be detected is pyrophosphoric acid generated by ligating a nick between two probes hybridized to a target DNA by a DNA ligase reaction. When two probes are hybridized to a target DNA with a nick therebetween, a DNA ligase can ligate the nick, and pyrophosphoric acid is released at this time. On the other hand, in the case where two probes are hybridized to a target DNA with not a nick but one or a few bases therebetween, or in a case where a probe is not hybridized, a DNA ligase reaction does not occur. Therefore, it is possible to detect a target DNA or a single nucleotide polymorphism in a target DNA by detecting pyrophosphoric acid.

In this manner, the invention relates to a method for detecting a target DNA and a method for detecting a single nucleotide polymorphism in a target DNA. The detection of a target DNA or a single nucleotide polymorphism is useful for genetic diagnosis.

The DNA ligase is an enzyme that ligates two DNA strands and is well known in this technical field. In the invention, an arbitrary DNA ligase can be used, and an appropriate DNA ligase can be obtained by utilizing a genetic engineering technique by those skilled in the art or as a commercially available product. Also, the target DNA is not limited, and a naturally-occurring product such as a biogenic product may be used, or a synthetic product such as a library may be used. The probe can be appropriately designed by those skilled in the art on the basis of the sequence of a target DNA to be detected or a target DNA containing a single nucleotide polymorphism to be detected.

In the case where pyrophosphoric acid generated by a DNA ligase reaction is detected, a DNA ligase and/or a target DNA may be immobilized on an electrically conductive support. For example, a DNA ligase and/or a target DNA may be immobilized on a surface of an electrode having a compound containing a boronic acid group immobilized thereon. Preferably, a DNA ligase is immobilized on an electrically conductive support or an electrode.

As a method for immobilizing a DNA polymerase or a DNA ligase on an electrically conductive support or an electrode, a peptide tag that specifically binds to a boronic acid group can be utilized. The present inventors have obtained a finding that a boronic acid group and an amino acid containing a hydroxy group in a side chain (a hydroxy group-containing amino acid) can bind to each other (see Reference Example 1). Therefore, by fusing a peptide tag containing such a hydroxy group-containing amino acid to a DNA polymerase or a DNA ligase, the DNA polymerase or the DNA ligase can be immobilized on an electrically conductive support or an electrode through the binding between the peptide tag and a boronic acid group. Alternatively, some types of DNA polymerases or DNA ligases comprise a molecule containing a lot of hydroxy group-containing amino acids. Such a DNA polymerase or a DNA ligase has binding affinity for a boronic acid group in itself, and therefore, by utilizing the affinity, it can be immobilized on an electrically conductive support.

The length and the composition (sequence) of the peptide tag are not particularly limited as long as the peptide tag contains an amino acid containing a hydroxy group in a side chain thereof, i.e., a hydroxy group-containing amino acid. As the hydroxy group-containing amino acid, serine (Ser, S), threonine (Thr, T), and tyrosine (Tyr, Y) are preferred, and the peptide tag can contain one or more types of these amino acids. The peptide tag preferably contains tyrosine as the hydroxy group-containing amino acid. In order for the peptide tag to have binding affinity for efficiently binding to a boronic acid group, it is necessary to allow two hydroxy groups to exist sterically close to each other to supply the hydroxy groups to a boronic acid group, and therefore, the peptide tag preferably contains two or more hydroxy group-containing amino acids. Further, in order to increase the number of combinations of two hydroxy groups which can be brought close to a boronic acid group, the peptide tag is preferably composed of 4 to 6 consecutive hydroxy group-containing amino acid residues. Alternatively, a peptide tag composed of 7 or more (e.g., 7 to 50) consecutive hydroxy group-containing amino acid residues is also possible. However, as the number of consecutive hydroxy group-containing amino acid residues is increased, the peptide tag may affect the formation of the conformation or activity of a DNA polymerase or a DNA ligase to be fused to the peptide tag. Accordingly, it is preferred to design the peptide tag in consideration of the effect.

Further, between two or more hydroxy group-containing amino acids, a non-hydroxy group-containing amino acid may be contained, or a non-hydroxy group-containing amino acid may be attached to a terminal end of hydroxy group-containing amino acids. For example, a peptide tag in which two residues at both terminal ends sandwiching two or more arbitrary amino acid residues are hydroxy group-containing amino acid residues can be used.

Further, it is known that in order to increase the efficiency of binding to a boronic acid group, a local pH at a site where the binding reaction between a boronic acid group and a peptide tag occurs is preferably basic. Therefore, it is preferred that in the peptide tag, 1 to 4 (preferably 1 to 3) basic amino acid residues are adjacent to the above-described consecutive hydroxy group-containing amino acids or inserted in the sequence of the consecutive hydroxy group-containing amino acids. Here, the basic amino acid refers to an amino acid selected from lysine (Lys, K), arginine (Arg, R), and tryptophan (Trp, W), and the peptide tag can contain one or more types of these amino acids. For example, as the peptide tag, a peptide tag composed of 4 to 6 amino acid residues in total including 3 to 5 hydroxy group-containing amino acid residues and 1 to 3 basic amino acid residues, or a peptide tag composed of 7 or more amino acid residues in total including 4 or more hydroxy group-containing amino acid residues and 1 to 3 basic amino acid residues can be exemplified.

Specific examples of the peptide tag are shown below.

(SEQ ID NO: 4)
Tyr-Tyr-Tyr-Tyr-Tyr-Tyr (6Y)
(SEQ ID NO: 5)
Ser-Ser-Ser-Ser-Ser-Ser-Trp (6SW)
(SEQ ID NO: 6)
Ser-Ser-Ser-Ser-Trp (4SW)
(SEQ ID NO: 7)
Tyr-Tyr-Tyr-Tyr-Tyr-Tyr-Trp (6YW)
(SEQ ID NO: 8)
Tyr-Tyr-Tyr-Tyr-Trp (4YW)
(SEQ ID NO: 9)
Tyr-Tyr-Tyr-Tyr-Tyr-Tyr-Gly-Gly (6Y2G)
(SEQ ID NO: 10)
Tyr-Tyr-Tyr-Tyr-Tyr-Tyr-Lys-Lys (6Y2K)
(SEQ ID NO: 11)
Tyr-Tyr-Tyr-Tyr-Tyr-Tyr-Gly-Lys (6YGK)
(SEQ ID NO: 12)
Tyr-Tyr-Tyr-Tyr-Trp-Tyr-Tyr (4YW2Y)
(SEQ ID NO: 13)
Tyr-Tyr-Tyr-Trp-Tyr-Tyr-Tyr (3YW3Y)
(SEQ ID NO: 14)
Tyr-Tyr-Tyr-Tyr-Tyr-Trp-Tyr (5YWY)
(SEQ ID NO: 15)
Tyr-Tyr-Tyr-Tyr-Tyr-Tyr-Lys-Lys-Trp (6Y2KW)
(SEQ ID NO: 16)
Tyr-Tyr-Tyr-Tyr-Tyr-Tyr-Trp-Lys-Lys (6YW2K)

In the invention, a peptide tag as described above is fused to a DNA polymerase or a DNA ligase, whereby a fusion protein is formed. Further, by fusing an arbitrary molecule (such as a protein, a polypeptide, or a peptide) desired to be immobilized on an electrically conductive support to the peptide tag, immobilization on an electrically conductive support can be achieved easily and efficiently.

The peptide tag can be fused to a DNA polymerase or a DNA ligase or other molecule at an arbitrary site thereof. However, it is preferred that the peptide tag is fused to a DNA polymerase or a DNA ligase or other molecule at a site apart from its active site or recognition site so that the peptide tag does not affect the conformation or activity of such a molecule.

Further, the peptide tag can be fused to a DNA polymerase or a DNA ligase or other molecule by a method or means known in this technical field. A method for preparing a fusion protein is well known in this technical field, and for example, a chemical synthesis method or a genetic recombination method can be used. In the case of a chemical synthesis method, according to a known peptide synthesis method, for example, by using a commercially available peptide synthesizer or a commercially available kit for peptide synthesis, a fusion protein can be prepared. In the case of a genetic recombination method, for example, a DNA encoding a DNA polymerase or a DNA ligase or other molecule and a DNA encoding a peptide tag are ligated to each other directly or through a linker sequence, the resulting fragment is inserted into a known vector such as a plasmid, the resulting vector is introduced into a host cell, and a fusion protein can be expressed in the host cell (see, for example, Reference Example 2). The peptide tag is preferably attached to the carboxyl terminal end and/or the amino terminal end of a DNA polymerase or a DNA ligase or other molecule. The carboxyl terminal end or the amino terminal end of a protein molecule often protrudes in a region capable of being in contact with a solvent as an end point of the molecule, and therefore, by attaching the peptide tag thereto, an effect on the formation of a correct three-dimensional structure of the protein can be suppressed, and also the inhibition of the function of the protein itself can be suppressed.

Further, in order to further decrease the possibility of inhibiting the function of a DNA polymerase or a DNA ligase or other molecule when attaching the peptide tag to the carboxyl terminal end and/or the amino terminal end of the DNA polymerase or DNA ligase or other molecule, it is preferred to attach a linker sequence composed of about several residues to the amino terminal side of the peptide tag in the case of attaching the peptide tag to the carboxyl terminal end, and to the carboxyl terminal side of the peptide tag in the case of attaching the peptide tag to the amino terminal end. The linker sequence to be inserted between the protein molecule and the peptide tag may be a linker sequence known in this technical field, and for example, a linker sequence composed of 1 to 20 amino acids, preferably 1 to 10 amino acids, more preferably 2 to 6 amino acids can be used. Specific examples of the linker sequence include a sequence composed of about 4 to 6 consecutive glycine residues which has an advantage in that the degree of freedom of conformation is high (see, for example, Reference Example 2).

As the vector to be used for expressing the fusion protein, any vector can be used as long as it is a known vector such as a plasmid, a phagemid, a virus-derived vector (an animal virus vector such as a retrovirus, adenovirus, or vaccinia virus vector, or an insect virus vector such as baculovirus vector), or an artificial chromosome. An expression vector for expressing a fusion protein can be obtained by ligating a DNA encoding a DNA polymerase or a DNA ligase or other molecule and a DNA encoding a peptide tag to a vector such that a fusion protein is expressed. In the expression vector, it is preferred to ligate a promoter, a transcription termination signal, if necessary an enhancer, a splicing signal, a poly-A addition signal, a selection marker, etc. The constructed expression vector is introduced into a host cell, whereby a transformant is produced. The host to be used for the transformation is not particularly limited as long as it can express and produce a fusion protein, and examples thereof include bacteria (such as E. coli), yeast, animal cells, and insect cells. The introduction of the expression vector into the host cell can be performed by a method known in this technical field such as an electroporation method, a calcium phosphate method, or a lipofection method. The fusion protein can be obtained by culturing the transformant, followed by isolation and purification from the culture.

As described above, a DNA polymerase or a DNA ligase or other molecule containing a peptide tag having binding affinity for a boronic acid group in its molecule can be formed.

A DNA polymerase or a DNA ligase having binding affinity for a boronic acid group, or a DNA polymerase or a DNA ligase containing a peptide tag having binding affinity for a boronic acid group in its molecule can be immobilized on an electrically conductive support through binding to a boronic acid group. For example, a solution containing such a DNA polymerase or a DNA ligase is added to a solution in which an electrically conductive support is placed, followed by incubation, whereby the DNA polymerase or the DNA ligase can be bound to a boronic acid group on the electrically conductive support. Alternatively, a solution of a DNA polymerase or a DNA ligase mixed in an aqueous solution such as a buffer with a near neutral pH is brought into contact with a compound containing a boronic acid group immobilized on an electrically conductive support, and then left at 5 to 40° C., preferably at room temperature for several minutes to several tens of minutes.

As described above, by fusing a peptide tag that specifically and spontaneously binds to a boronic acid group to a DNA polymerase or a DNA ligase or other molecule to be immobilized, the DNA polymerase or DNA ligase or other molecule can be stably immobilized in an arbitrary orientation on an electrically conductive support through the binding between a boronic acid group and the peptide tag using a compound containing the boronic acid group as a linker.

On the other hand, the target DNA or RNA may be immobilized on a solid support different from an electrically conductive support. The solid support to be used is not particularly limited as long as it is a solid support generally used for immobilizing a DNA or an RNA. Specifically, it is preferably a solid support which is insoluble in water and does not melt during denaturation by heating. Examples of a material thereof include metals such as gold, silver, copper, aluminum, tungsten, molybdenum, chromium, platinum, titanium, and nickel; alloys such as stainless steel, hastelloy, inconel, monel, and duralumin; silicon; glass materials such as glass, quartz glass, fused quartz, synthetic quartz, alumina, sapphire, ceramics, forsterite, and photosensitive glass; plastics such as polyester resins, polystyrene, polyethylene resins, polypropylene resins, ABS resins (acrylonitrile butadiene styrene resins), nylon, acrylic resins, fluororesins, polycarbonate resins, polyurethane resins, methylpentene resins, phenol resins, melamine resins, epoxy resins, and vinyl chloride resins; agarose, dextran, cellulose, polyvinyl alcohol, nitrocellulose, chitin, and chitosan. Also, the form of the solid support is not particularly limited, and a support formed of a plane (e.g., a titer plate, or a porous or macroporous array), a flat plate, a film, a tube, and a particle can be exemplified. Further, by using a metal bead or a magnetic bead which has been magnetized or can be magnetized as the particle, a separation treatment or the like can be automatized and accelerated, or the efficiency thereof is increased. In the case where a particle is used as the solid support, the diameter of the particle is generally 50 μm or less, for example, from 1.0 μm to 3.0 μm.

In an embodiment, in each of a plurality of reaction cells, a target DNA or RNA immobilized on a solid support as described above is placed, a DNA complementary strand synthesis reaction with a DNA polymerase is performed, an electrical change corresponding to each reaction cell is measured, and pyrophosphoric acid generated is detected. As the target DNA or RNA, a plurality of the same type may be used or a plurality of types may be used.

Further, the invention provides a device for electrically detecting pyrophosphoric acid, characterized by including an electrically conductive support having a compound containing a boronic acid group immobilized thereon. In the device of the invention, the compound containing a boronic acid group may be immobilized on the electrically conductive support through a functional group such as a head group, or may be immobilized by forming an electrically conductive support (immobilization base material) by crosslinking the compound containing a boronic acid group with a crosslinking molecule. The form of the device is not particularly limited as long as the device includes the immobilized compound containing a boronic acid group, and may be the form of an electrically conductive support such as a form of a base plate (such as a protein chip) or a particle (such as a nanoparticle), or the form of an immobilization base material such as a form of a membrane, a film, or a gel.

FIG. 4 shows one example of a device including an electrically conductive support further having a DNA polymerase or a DNA ligase immobilized thereon as a molecule, and one example of detection of pyrophosphoric acid using the device. On an electrically conductive support 401, a compound containing a boronic acid group 402 is immobilized (FIGS. 4A and 4B). A DNA polymerase or DNA ligase 404 can be immobilized on the electrically conductive support 401 through binding between a peptide tag 403 and the compound containing a boronic acid group 402. When pyrophosphoric acid is released by a reaction of the DNA polymerase or DNA ligase 404 (405), the released pyrophosphoric acid comes into contact with the compound containing a boronic acid group 402 on the electrically conductive support 401 (406), whereby an electrical change occurs.

In the device of the invention, a compound containing a boronic acid group or other molecule can be immobilized while controlling the orientation thereof and/or as a monolayer, and therefore, the device is useful for various analyses.

Further, the invention provides a sensor for detecting pyrophosphoric acid including an electrically conductive support having a compound containing a boronic acid group immobilized thereon, and a unit for detecting an electrical change. As the detection unit, a measurement device that can measure a minute voltage change as described above, for example, an electrical detection unit such as an FET can be used.

A field-effect transistor (FET) is a technique widely used in a sensor utilizing electrical detection. The principle is as follows. When an electric current flowing from a source electrode to a drain electrode is controlled by a gate electrode serving as a third electrode and an intermolecular interaction is allowed to occur on a surface of the gate electrode, an electric charge of the gate electrode is changed, and therefore, electric current response is changed.

When a reaction that causes charge transfer takes place on a sensor chip, an FET can achieve detection with high sensitivity. However, the reaction should take place at a site as close as possible to the surface of the gate electrode, and a detectable distance is supposed to be several nanometers. Therefore, as disclosed in the present invention, when a device in which a molecule is immobilized while controlling the orientation and keeping the height at the same level at high precision is used as a gate electrode, the arrangement of the molecule can be controlled within the detection limit of several nanometers. Accordingly, the device of the invention is useful as a sensor utilizing a highly sensitive FET.

The device and the sensor of the invention can be used in DNA sequencing, detection of a target DNA or RNA, or detection of a single nucleotide polymorphism on the basis of the detection of pyrophosphoric acid as described above.

The advantages of the invention are as follows: (i) immediate measurement can be performed only with one enzyme which plays a role in the process of releasing pyrophosphoric acid; (ii) since pyrophosphoric acid is directly detected using released pyrophosphoric acid as a substrate without undergoing other enzymatic reactions, the amount of released pyrophosphoric acid and the intensity of detection signal are in a linear relationship; and (iii) since a boronic acid group with which an electrode or the like is modified captures pyrophosphoric acid, and at the same time, emits an electrical signal, the amount of released pyrophosphoric acid can be quantitatively measured almost without being affected by the shape of a reaction cell or the amount of a solution.

Further, a boronic acid group to be used in the invention can be introduced into various solid supports through a variety of functional groups, and therefore, a wide variety of devices can be used for detection or measurement. Phenylboronic acid which is one of the compounds containing a boronic acid group has been already utilized as a molecule incorporated into a polymer capable of spontaneously producing a film or a gel. By utilizing such a molecule, also on a base plate made of a material which is extremely stable and is difficult to newly introduce a chemical bond such as a ceramic or a glass, a voltage change measurement system can be constructed by coating the base plate with a film composed of a polymer containing phenylboronic acid (i.e., a pyrophosphoric acid-responsive film).

The invention can be applied to all systems in which pyrophosphoric acid is generated as a reaction product as a result of various chemical processes. In many biochemical processes, enzymes act on a nucleotide triphosphate and degrades it into a nucleotide monophosphate and a pyrophosphoric acid, and high energy is obtained in this process. Accompanying this, various biosynthesis reactions are coupled in many cases. That is, this reaction is coupled with another reaction of the same enzyme or is coupled with a reaction of another enzyme in some cases. Due to this, by appropriately selecting an enzymatic system and measuring the release of pyrophosphoric acid under the coexistence of a plurality of enzymes as needed, it is possible to indirectly detect the occurrence of other reaction. The invention provides a technique for diachronically observing the progress of such biochemical processes.

### EXAMPLES

Hereinafter, specific examples of embodiments of the invention will be described with reference to drawings. However, it should be noted that these Examples are merely illustrative for embodying the invention and do not limit the invention.

### Example 1

### Preparation of Solid Support Having Phenylboronic Acid Group Bound Thereto

A gold base plate (10×10 mm, thickness: 100 mm, gold was sputtered on silicon nitride) was dipped in a washing solution (a piranha solution) obtained by mixing concentrated sulfuric acid and an aqueous solution of hydrogen peroxide at 3:1 and washing was performed for 1 hour while sometimes stirring the solution.

4-Mercaptophenyl boronic acid was dissolved in ethanol at 10 mM, and the above gold thin plate was dipped therein and left to stand for 12 hours, whereby a gold base plate having 4-mercaptophenyl boronic acid immobilized thereon was obtained.

### Example 2

### Measurement of Minute Voltage Change when Adding Pyrophosphoric Acid in Gold Base Plate Treated with Phenylboronic Acid and Untreated Gold Base Plate

After preparing and washing a gold base plate by a method shown in Example 1, each of the gold base plate treated with 4-mercaptophenyl boronic acid and the untreated gold base plate was dipped in phosphate buffered saline (10 mM NaH₂PO₄/Na₂HPO₄, 150 mM NaCl, pH 7.4) and equilibrated for 1 hour.

Each of the gold base plates was connected to a gate electrode of a field-effect transistor (FET), and separately dipped in 100 μL of phosphate buffered saline and equilibrated for an additional 30 minutes. Then, from the solution, the ground connection was made through abridge filled with a gel, and the measurement of a minute voltage change was started.

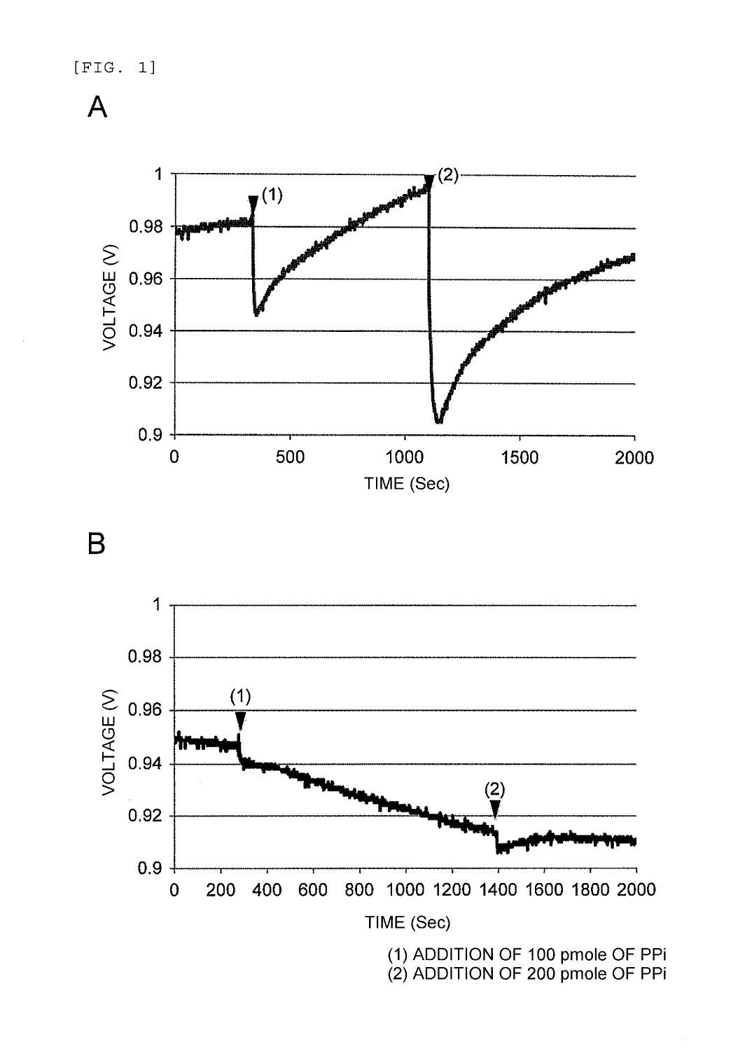

A voltage change curve continued to decrease slightly at the beginning in both cases of using the gold base plate treated with 4-mercaptophenyl boronic acid and the untreated gold base plate. Thereafter, pyrophosphoric acid was added three times at intervals of 800 to 1000 seconds at (1) 100 pmol or (2) 200 pmol, respectively (FIG. 1).

In the case of using the gold base plate modified with 4-mercaptophenyl boronic acid (FIG. 1A), a change in voltage of several tens of millivolts was observed at each time of adding pyrophosphoric acid, and the degree of change varied depending on the amount of pyrophosphoric acid added. On the other hand, in the case of using the unmodified gold base plate (FIG. 1B), a significant response to the addition of pyrophosphoric acid was not observed.

### Example 3

### Measurement of Pyrophosphoric Acid Generated when Adding and Reacting Deoxynucleotide Triphosphate with DNA Polymerase/Substrate DNA Complex Using Gold Base Plate Treated with Phenylboronic Acid

A pretreatment of a gold base plate was performed in the same manner as the procedure for the gold base plate treated with 4-mercaptophenyl boronic acid in Example 2.

Each gold base plate was connected to a gate electrode of a field-effect transistor (FET), and separately dipped in 90 μL of phosphate buffered saline and equilibrated for an additional 30 minutes.

In a DNA polymerase to be used, a sequence of a peptide tag that specifically recognizes and binds to phenylboronic acid was integrated in its molecule. Specifically, according to a procedure described in Reference Example 2, a plasmid encoding a DNA polymerase in place of luciferase was constructed.

To a solution in which the gold base plate was dipped, 10 μL of a 30 μM peptide-tagged DNA polymerase mixed previously with a template DNA was added, followed by incubation at 25° C. for 5 minutes. Thereafter, the supernatant was completely removed, and immediately thereafter, 100 μL of phosphate buffered saline was added thereto. By doing this, only the DNA polymerase bound to the gold base plate modified with phenylboronic acid through the peptide tag was left on the base plate, and pyrophosphoric acid released by the DNA polymerase molecule can be effectively and immediately detected. From the solution, the ground connection was made through a bridge filled with a gel, the base plate was connected to the gate electrode of the FET, and the measurement of a minute voltage change on the base plate was started.

The sequences (SEQ ID NOS: 1 and 2) of the template DNA are shown below.

(SEQ ID NO: 1)
5′-AAAAAAAAAAAAAAAAAAAA
(SEQ ID NO: 2)
3′-TTTTTTTTTTTTTTTTTTTTGGGGGGGGGGCCCCCCCCCCAAAAAAA
AAA

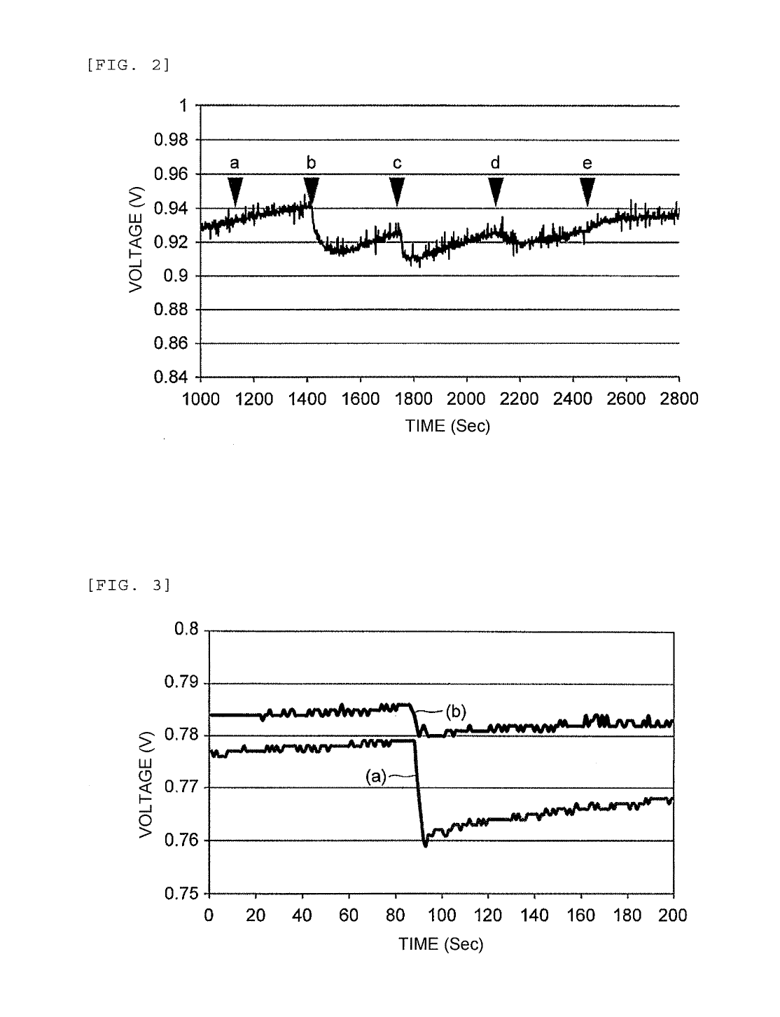

As shown in FIG. 2, it was confirmed that by adding (a) dATP, which is not a substrate of this sequence, a change was not observed in the sensorgram. As the substrates for DNA synthesis, (b) dCTP, (c) dGTP, and (d) dTTP were sequentially added. At this point, the reaction should have been completed, and therefore, (e) dNTP was added as a negative control in the end. Only when a base complementary to the sequence was added, a change was observed in the sensorgram. As for the amount of a deoxyribonucleic acid as a substrate added at this time, it was added to give a final concentration of 0.05 mM.

### Example 4

### Measurement of Pyrophosphoric Acid Generated when Adding and Reacting Deoxynucleotide Triphosphate with DNA Polymerase/Substrate DNA Complex Using Gold Base Plate Treated with Phenylboronic Acid

A pretreatment of a gold base plate was performed in the same manner as the procedure for the gold base plate treated with 4-mercaptophenyl boronic acid in Example 2.

A gold base plate modified with phenylboronic acid having an enzyme/substrate bound thereto was produced in the same manner as the procedure for immobilizing a peptide-tagged DNA polymerase on a base plate along with a template DNA in Example 3. Further, the other experimental conditions were also the same as those in Example 3.

At this time, experiments were performed by separately using two types of template DNAs, whose sequences are shown below. A template (a) is the same template DNA as in Example 3 and comprises single-base repeats (10 bases long) (SEQ ID NOS: 1 and 2), on the other hand, a template (b) comprises single-base repeats (3 bases long) (SEQ ID NOS: 1 and 3).

(a)
(SEQ ID NO: 1)
5′-AAAAAAAAAAAAAAAAAAAA
(SEQ ID NO: 2)
3′-TTTTTTTTTTTTTTTTTTTTGGGGGGGGGGCCCCCCCCCCAAAAAAA
AAA
(b)
(SEQ ID NO: 1)
5′-AAAAAAAAAAAAAAAAAAAA
(SEQ ID NO: 3)
3′-TTTTTTTTTTTTTTTTTTTTGGGCCCAAAGGGCCCAAAGGGCCCAAA
GGG

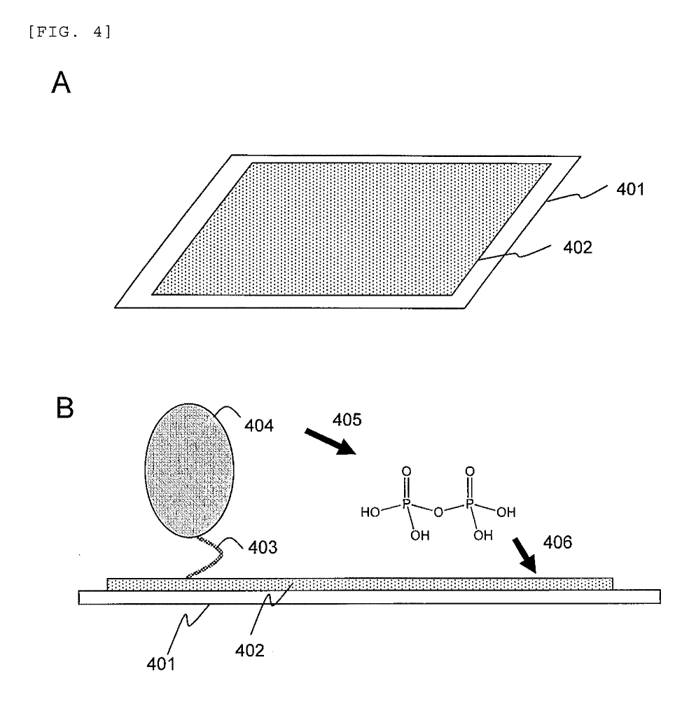

For each template, dCTP, which is a substrate to enable a reaction, was added to give a final concentration of 0.05 mM. The result is shown in FIG. 3 as a sensorgram.

The respective voltage change amounts in the sensorgrams varied depending on the number of consecutive guanine bases immediately downstream of the primer in the template DNA used. It is supposed that as the number of consecutive bases is larger, the amount of incorporation of dCTP is increased, and therefore, the amount of released pyrophosphoric acid is also increased according to the increase. Also shown in FIG. 3B, in the case of the template (a) which comprises single-base repeats (10 bases long), the amount of change was about 20 mA. However, in the case of the template (b) which comprises single-base repeats (3 bases long), the amount of change was about 6 mA.

### Reference Example 1

### Peptide Tag Having Binding Affinity for Boronic Acid

As one example of a polymer containing a boronic acid group, poly((2-methacryloyloxyethyl phosphorylcholine)-co-(n-butyl methacrylate)-co-(p-vinylphenylboronic acid)) (hereinafter abbreviated as PMBV) was obtained by polymerization such that the amount of a 2-methacryloyloxyethyl phosphorylcholine group was 60 mol %, the amount of n-butyl methacrylate was 20 mol %, and the amount of p-vinylphenylboronic acid was 20 mol %.

As another example of a polymer containing a boronic acid group, poly((2-methacryloyloxyethyl phosphorylcholine)-co-(dimethylaminoethyl methacrylate)-co-(p-vinylphenylboronic acid)) (hereinafter abbreviated as PMDV) was prepared such that the amount of a 2-methacryloyloxyethyl phosphorylcholine group was 60 mol %, the amount of dimethylaminoethyl methacrylate was 20 mol %, and the amount of p-vinylphenylboronic acid was 20 mol %.

This PMBV or PMDV was dissolved in phosphate buffered saline (PBS: 10 mM NaH₂PO₄/Na₂HPO₄, 150 mM NaCl, pH 7.3) at 0.25 mg/mL, and 200 μL of each of the resulting solutions was dispensed in a 96-well plate.

In each well containing the above solution, an oligopeptide having the following sequence (in PBS) was dispensed at 0.05 mM per well, and mixed with the solution in the well.

(SEQ ID NO: 4)
Tyr-Tyr-Tyr-Tyr-Tyr-Tyr (6Y)
(SEQ ID NO: 5)
Ser-Ser-Ser-Ser-Ser-Ser-Trp (6SW)
(SEQ ID NO: 6)
Ser-Ser-Ser-Ser-Trp (4SW)
(SEQ ID NO: 7)
Tyr-Tyr-Tyr-Tyr-Tyr-Tyr-Trp (6YW)
(SEQ ID NO: 8)
Tyr-Tyr-Tyr-Tyr-Trp (4YW)
(SEQ ID NO: 9)
Tyr-Tyr-Tyr-Tyr-Tyr-Tyr-Gly-Gly (6Y2G)
(SEQ ID NO: 10)
Tyr-Tyr-Tyr-Tyr-Tyr-Tyr-Lys-Lys (6Y2K)
(SEQ ID NO: 11)
Tyr-Tyr-Tyr-Tyr-Tyr-Tyr-Gly-Lys (6YGK)
(SEQ ID NO: 12)
Tyr-Tyr-Tyr-Tyr-Trp-Tyr-Tyr (4YW2Y)
(SEQ ID NO: 13)
Tyr-Tyr-Tyr-Trp-Tyr-Tyr-Tyr (3YW3Y)
(SEQ ID NO: 14)
Tyr-Tyr-Tyr-Tyr-Tyr-Trp-Tyr (5YWY)
(SEQ ID NO: 15)
Tyr-Tyr-Tyr-Tyr-Tyr-Tyr-Lys-Lys-Trp (6Y2KW)
(SEQ ID NO: 16)
Tyr-Tyr-Tyr-Tyr-Tyr-Tyr-Trp-Lys-Lys (6YW2K)

The well plate was shaken at 25° C. for 2 hours (700 rpm, TAITEC MBR-022UP). After the shaking, a fluorescent dye alizarin red S that specifically binds to a phenylboronic acid group in PMBV or PMDV was dispensed in each well at 0.05 mM and mixed with the solution in the well. The well plate was shaken at 25° C. for 1 hour (700 rpm, TAITEC MBR-022UP).

In order to find out how much alizarin red S could bind to a phenylboronic acid group, a fluorescence intensity derived from alizarin red S was measured. By using a fluorospectrometer Gemini XS of Molecular Devices, Inc., each sample was excited at an excitation wavelength of 495 nm, and a fluorescence emission intensity was continuously measured at 10 nm intervals in a range of 520 nm to 640 nm, and the maximum value was determined as the fluorescence intensity.

The amount of binding between the peptide and the phenylboronic acid group was obtained from the maximum fluorescence intensity derived from alizarin red S in each of the cases where the peptide was not added and the respective peptides were added. That is, as the fluorescence intensity derived from alizarin red S is lower, the peptide inhibits the binding of alizarin red S to the phenylboronic acid group more, and therefore, the amount of binding between the peptide and the phenylboronic acid group is large. From the result, it was found that all the oligopeptides bind to the phenylboronic acid group. This result was also confirmed by separately performed surface plasmon resonance measurement.

### Reference Example 2

### Construction of Plasmid Encoding Luciferase Tagged with Peptide Having Binding Affinity for Phenylboronic Acid and Expression Thereof

By using pURE2 Luc plasmid which is a luciferase (hereinafter abbreviated as Luc) expression plasmid as a template, peptide tags of 6Y2K (SEQ ID NO: 10) and 6YW (SEQ ID NO: 7) were introduced into a protein molecule such that the peptide tags can be attached to the C-terminal end via 4 glycine residues. As an introduction method into the plasmid, an inverse PCR method was used. In the inverse PCR method, primers are selected such that the primers face away from each other on the basis of a sequence introduction site of a circular DNA. At this time, to the primers, a sequence to be introduced is previously attached as anchor sequences 1 and 2. By carrying out a PCR reaction under this condition, a linear double-stranded DNA in which the anchor sequences 1 and 2 are attached to the terminal ends is produced. This DNA fragment is ligated by a self-ligation reaction, whereby a circular double-stranded DNA into which the anchor sequences have been introduced at a desired site can be obtained.

As for the primers, a sequence of 5′-TATTATTATAAAAAATAGCATATGAAGCTTTAGCATAACCCCTT-3′ (SEQ ID NO: 17) as a forward primer for 6Y2K and a sequence of 5′-TATTATTATTGGTAGCATATGAAGCTTTAGCATAACCCCTT-3′ (SEQ ID NO: 18) as a forward primer for 6YW were used, and a sequence of 5′-ATAATAATAACCACCACCACCCAATTTGGACTTTCCGCCCT-3′ (SEQ ID NO: 19) was used as a common reverse primer.

An inverse PCR reaction solution was prepared by adding 20 fmol of the template plasmid pURE2 Luc, 15 pmol of the forward primer, 15 pmol of the reverse primer, 2.5 U of KOD-Plus DNA polymerase (TOYOBO Co., Ltd.), 5 μL of 10×KOD buffer, and 5 μL of 10 mM dNTP, and adjusting the total volume to 50 μL with dH₂O. An amplification reaction was performed as follows: after performing a reaction at 95° C. for 1 minute, a cycle reaction consisting of 98° C. for 30 seconds, 55° C. for 1 minute, and 68° C. for 4 minutes was repeated 30 times, followed by cooling to 4° C.

The sample in an amount of 50 μL was collected, 1 μL of DpnI (20 U/μL, NEB Corporation) was added thereto, and a reaction was allowed to proceed at 37° C. for 1 hour to degrade the template plasmid. To the solution after the reaction, 150 μL of ethanol and 5 μL of NaOAC were added, and the resulting mixture was centrifuged at 15000 rpm for 30 minutes at 4° C. Then, the supernatant was discarded (ethanol precipitation).

The precipitate was dissolved in 10 μL of dH₂O, 1 μL of T4 kinase (TaKaRa Co., Ltd.) and 14 μL of Ligation high (TOYOBO Co., Ltd.) were mixed therein, and a reaction was allowed to proceed at 37° C. for 1 hour. Then, 5 μL of Ligation high was further added thereto, and a reaction was allowed to proceed at 4° C. for 1 hour to effect a self-ligation reaction.

5 μL of the self-ligation reaction mixture was mixed with 50 μL of competent cells JM109 (TaKaRa Co., Ltd.), and the resulting mixture was left to stand on ice for 10 minutes, followed by a heat shock reaction at 42° C. for 45 seconds to effect transformation of E. coli. The solution was plated on an LB-amp (LB broth, Invitrogen, Inc., agar, Wako Pure Chemical Industries Ltd., ampicillin, Sigma, Inc.) plate, and the cells were cultured overnight at 37° C., whereby E. coli colonies were formed. A few colonies were picked up and cultured overnight in 20 mL of LB-amp culture medium, and then, the cells were collected. The plasmids were extracted and purified using a Plasmid Miniprep System (Marligen Biosciences, Inc.). The sequencing of the purified plasmids was performed, and a plasmid in which the introduction of the sequence was confirmed was stored.

1 μL of 100 ng/μL of the plasmid (Luc6Y2K) into which the 6Y2K sequence (SEQ ID NO: 10) was introduced at a C-terminal side of Luc, and 1 μL of 100 ng/μL of the plasmid (Luc6YW) into which the 6YW sequence (SEQ ID NO: 7) was introduced were added to 50 μL of competent cells BL21 (Novagen, Inc.) for protein expression, and the resulting mixture was left to stand at 4° C. for 10 minutes, followed by a heat shock reaction at 42° C. for 30 seconds to effect transformation. The solution was plated on an LB-amp (LB broth, Invitrogen, Inc., agar, Wako Pure Chemical Industries Ltd., ampicillin, Sigma, Inc.) plate, and the cells were cultured overnight at 37° C., whereby E. coli colonies were formed. A few colonies were picked up and cultured overnight in 2 L of LB-amp culture medium at 25° C., and then, the cells were collected.

The cells were suspended in a cell membrane disruption solution, and the resulting mixture was mixed by gently rotating at room temperature for 10 minutes. After centrifugation (30,000 rpm, 30 minutes), the supernatant was collected. Since both of Luc-6Y2K and Luc-6YW had a Strep-tag sequence at the N-terminal side, affinity chromatography purification utilizing an avidin-biotin interaction was performed using AKTA explorer 10S system. The supernatant obtained by the centrifugation was filtered through a 0.1 μm filter, and the resulting filtrate was charged to StrepTrap HP 5 mL (GE Healthcare Japan Corporation) at a flow rate of 5 mL/min to adsorb the sample. After adsorbing the sample, the column was washed with 10 mL of phosphate buffered saline (10 mM NaH₂PO₄/Na₂HPO₄, 150 mM NaCl, pH 7.3). Then, the target protein adsorbed to the column was eluted using phosphate buffered saline in which desthiobiotin (Sigma-Aldrich Co., Ltd.) was dissolved. The eluted sample was subjected to SDS polyacrylamide electrophoresis for each fraction, and it was confirmed that the target protein was expressed and the other contaminant proteins were removed.

Note that the present invention is not limited to the above-described embodiments, but includes various modifications. For example, the above-described embodiments have been described in detail so as to assist the understanding of the present invention, and the invention is not always limited to embodiments having all the described constituent elements. Further, it is possible to replace a part of constituent elements of an embodiment with constituent elements of another embodiment, and it is also possible to add a constituent element of an embodiment to a constituent element of another embodiment. Further, regarding a part of a constituent element of each embodiment, it is possible to perform addition, deletion, or replacement using other constituent elements.

All publications, patents, and patent applications cited herein are hereby incorporated by reference in their entirety.

### REFERENCE SIGNS LIST

401: electrically conductive support
402: compound containing boronic acid group
403: peptide tag
404: DNA polymerase or DNA ligase
405: release of pyrophosphoric acid
406: contact between boronic acid group and pyrophosphoric acid

### SEQUENCE LISTING FREE TEXT

SEQ ID NOS: 1 to 3: Artificial Sequences (synthetic oligonucleotides)

SEQ ID NOS: 4 to 16: Artificial Sequences (synthetic oligopeptides)

SEQ ID NOS: 17 to 19: Artificial Sequences (synthetic oligonucleotides)

1. A method for electrically detect pyrophosphoric acid, comprising:
a step of preparing an electrically conductive support having a compound containing a boronic acid group immobilized thereon;
a step of bringing a test substance into contact with a boronic acid group on the electrically conductive support;
a step of measuring an electrical change of the electrically conductive support; and
a step of detecting pyrophosphoric acid on the basis of the electrical change.

2. The method according to claim 1, wherein the compound containing a boronic acid group is at least one compound selected from the group consisting of a phenylboronic acid derivative, a methylboronic acid derivative, and a propenylboronic acid derivative.

3. The method according to claim 1, wherein the compound containing a boronic acid group is a water-soluble polymer molecule containing a boronic acid group.

4. The method according to claim 1, wherein the compound containing a boronic acid group is a molecule containing a phenylboronic acid group and a thiol group.

5. The method according to claim 1, wherein the electrically conductive support contains a noble metal.

6. The method according to claim 1, wherein the electrical change is measured by a voltage change measurement device connected to the electrically conductive support through an electrical conductor.

7. The method according to claim 6, wherein the measurement device is a measurement device having a field-effect transistor (FET) in a circuit for measuring a minute voltage change.

8. A method for sequencing a target DNA, comprising:
a step of detecting pyrophosphoric acid generated by DNA complementary strand synthesis with a DNA polymerase using a target DNA as a template according to the method according to claim 1; and
a step of determining the type of base incorporated in the DNA complementary strand synthesis on the basis of the result of detection of pyrophosphoric acid.

9. The method according to claim 8, wherein the DNA polymerase and/or the target DNA are/is immobilized on a surface of the electrically conductive support.

10. The method according to claim 9, wherein the DNA polymerase to be immobilized on the electrically conductive support is a DNA polymerase containing a peptide tag having binding affinity for a boronic acid group in its molecule.

11. The method according to claim 8, wherein the target DNA or RNA is immobilized on a surface of a solid support which is different from the electrically conductive support.

12. A method for detecting a target DNA or RNA, comprising:
a step of detecting pyrophosphoric acid generated by DNA complementary strand synthesis with a DNA polymerase from a probe hybridized to the target DNA or RNA according to the method according to claim 1;
a step of detecting the hybridization to the target DNA or RNA on the basis of the result of detection of pyrophosphoric acid.

13. A method for detecting a target DNA or detecting a single nucleotide polymorphism in a target DNA, comprising:
a step of detecting pyrophosphoric acid generated by ligating a nick between two probes hybridized to the target DNA by a DNA ligase reaction according to the method according to claim 1; and
a step of detecting the hybridization of the two probes to the target DNA on the basis of the result of detection of pyrophosphoric acid.

14. A device for electrically detecting pyrophosphoric acid, characterized by comprising an electrically conductive support having a compound containing a boronic acid group immobilized thereon.

15. The device according to claim 14, wherein on the electrically conductive support, further a DNA polymerase is immobilized.

16. The method according to claim 2, wherein the compound containing a boronic acid group is a water-soluble polymer molecule containing a boronic acid group.

17. The method according to claim 2, wherein the compound containing a boronic acid group is a molecule containing a phenylboronic acid group and a thiol group.

18. The method according to claim 3, wherein the compound containing a boronic acid group is a molecule containing a phenylboronic acid group and a thiol group.

19. The method according to claim 2, wherein the electrically conductive support contains a noble metal.

20. The method according to claim 3, wherein the electrically conductive support contains a noble metal.


SEQUENCE LISTING (19 sequences)

SEQ ID NO: 1 - DNA, 20 bp, Artificial
  Synthetic oligonucleotide
aaaaaaaaaaaaaaaaaaaa

SEQ ID NO: 2 - DNA, 50 bp, Artificial
  Synthetic oligonucleotide
ttttttttttttttttttttggggggggggccccccccccaaaaaaaaaa

SEQ ID NO: 3 - DNA, 50 bp, Artificial
  Synthetic oligonucleotide
ttttttttttttttttttttgggcccaaagggcccaaagggcccaaaggg

SEQ ID NO: 4 - protein, 6 aa, Artificial
  synthetic oligopeptide
YYYYYY

SEQ ID NO: 5 - protein, 7 aa, Artificial
  synthetic oligopeptide
SSSSSSW

SEQ ID NO: 6 - protein, 5 aa, Artificial
  synthetic oligopeptide
SSSSW

SEQ ID NO: 7 - protein, 7 aa, Artificial
  synthetic oligopeptide
YYYYYYW

SEQ ID NO: 8 - protein, 5 aa, Artificial
  synthetic oligopeptide
YYYYW

SEQ ID NO: 9 - protein, 8 aa, Artificial
  synthetic oligopeptide
YYYYYYGG

SEQ ID NO: 10 - protein, 8 aa, Artificial
  synthetic oligopeptide
YYYYYYKK

SEQ ID NO: 11 - protein, 8 aa, Artificial
  synthetic oligopeptide
YYYYYYGK

SEQ ID NO: 12 - protein, 7 aa, Artificial
  synthetic oligopeptide
YYYYWYY

SEQ ID NO: 13 - protein, 7 aa, Artificial
  synthetic oligopeptide
YYYWYYY

SEQ ID NO: 14 - protein, 7 aa, Artificial
  synthetic oligopeptide
YYYYYWY

SEQ ID NO: 15 - protein, 9 aa, Artificial
  synthetic oligopeptide
YYYYYYKKW

SEQ ID NO: 16 - protein, 9 aa, Artificial
  synthetic oligopeptide
YYYYYYWKK

SEQ ID NO: 17 - DNA, 44 bp, Artificial
  synthetic oligonucleotide
tattattataaaaaatagcatatgaagctttagcataacccctt

SEQ ID NO: 18 - DNA, 41 bp, Artificial
  synthetic oligonucleotide
tattattattggtagcatatgaagctttagcataacccctt

SEQ ID NO: 19 - DNA, 41 bp, Artificial
  synthetic oligonucleotide
ataataataaccaccaccacccaatttggactttccgccct